(19) United States
(12) Patent Application Publication
(10) Pub. No.: US 2019/0070157 A1
(43) Pub. Date: Mar. 7, 2019

(54) HYPERBARIC SOLUTION INJECTION OF LEVOBUPIVACAINE HYDROCHLORIDE COMPRISING LEVOBUPIVACAINE HYDROCHLORIDE

(71) Applicant: NEON LABORATORIES LIMITED, Mumbai (IN)

(72) Inventors: Neeta JOSHI, Mumbai (IN); Rahul NARKHEDE, Mumbai (IN)

(21) Appl. No.: 16/085,405

(22) PCT Filed: Apr. 1, 2016
(86) PCT No.: PCT/IN2016/050101

§ 371 Date: Sep. 14, 2018

(30) Foreign Application Priority Data
Jul. 13, 2015 (IN) 2641/MUM/2015

(51) Int. Cl. A61K 31/445; A61K 9/00; A61K 9/08; A61K 47/26; A61K 47/12; A61K 47/02

(52) U.S. Cl. CPC A61K 31/445; A61K 9/0019; A61K 47/02; A61K 47/26; A61K 47/12; A61K 9/08

(57) ABSTRACT

Disclosed herein is Hyperbaric solution for injection of Levobupivacaine Hydrochloride which comprises Levobupivacaine Hydrochloride; base/acid to adjust the pH and baricity adjuster to modify Baricity of the solution injection.

### TECHNICAL FIELD

The present invention provides hyperbaric injection solution of pharmaceutically acceptable salt of Levobupivacaine. More particularly the invention provides a hyperbaric solution injection of Levobupivacaine Hydrochloride comprising Levobupivacaine Hydrochloride; a baricity adjuster, a base/acid to adjust the pH with water as a vehicle. The invention further relates to process of preparation of a stable hyperbaric injection of Levobupivacaine Hydrochloride.

### BACKGROUND AND PRIOR ART

Levobupivacaine is a local anaesthetic drug belonging to the amino amide group. It is the S-enantiomer of Bupivacaine. Levobupivacaine and its use in solution, for infusion or injection into the epidural or spinal space, or for administration by any of the conventional means for obtaining a nerve or field block, is first disclosed in EP0821588 B1. Currently available (Marketed) formulation of Levobupivacaine Hydrochloride injection is a sterile solution of Levobupivacaine Hydrochloride in water for injections which is isobaric solution. Bupivacaine Hyperbaric solution is also available in market.

Levobupivacaine, the pure S (−)-enantiomer of Bupivacaine, emerged as a safer alternative for regional anesthesia than its racemic form. Levobupivacaine has demonstrated less affinity and strength of depressant effects onto myocardial and central nervous vital centres in pharmacodynamic studies, and has superior pharmacokinetic profile. Levo-enatiomer of bupivacaine appeared to have a safer pharmacological profile than its dextro-partner.

Levobupivacaine had a lower risk of cardiovascular and CNS toxicity than bupivacaine in animal studies.

CelltechGroup Company has launched Levobupivacaine under the trade name ‘Chirocaine’ in USA for use in surgical and obstetric surgery as local anesthesia and for postoperative pain. Experiments show that Levobupivacaine has better security then Bupivacaine and less central nervous system and cardiac toxicity. There available very few literature on the injectable solutions of levobupivacaine as mentioned below.

CN1628665 discloses Levobupivacaine freeze dried injection and its preparation method. The freeze dried injection comprises pharmaceutically acceptable salt of Levobupivacaine and freeze dryable adjuvant, wherein each preparation unit contains Levobupivacaine pharmaceutically acceptable salt 10-300 mg. The freeze dryable adjuvant includes diluent, isotonic conditioning agent, pH modifier.

Freshly prepared Levobupivacaine Hyperbaric solutions with glucose is reported in an article titled “Clinical Characteristics of Spinal Levobupivacaine: Hyperbaric Compared with Isobaric Solution”. This article concludes hyperbaric levobupivacaine solutions are more predictable for sensory block level and more effective for surgical procedures with lower abdominal approach; however, optimal dosage needs further investigation.

Levobupivacaine Hyperbaric solution gravitates to dependent areas. Hyperbaric solutions gravitate to the thoracic kyphosis in the supine patient, therefore assuring an adequate level of spinal anesthesia, which is T-6 in the average patient.

Hyperbaric solutions have a greater specific gravity than the cerebrospinal fluid often making the spread of anaesthesia more predictable with greater spread in the direction of gravity.

For surgical procedure performed on patients who are not in the supine position, the baricity of the local anaesthetic solution and gravity are employed to direct the local anaesthetic towards the spinal nerves innervating the surgical site.

Hyperbaric Levobupivacaine solution will provide a more rapid onset and greater spread of anasthesia but a shorter duration of anaesthesia and analgesia. Thus this solution is primarily useful for abdominal surgery procedures of limited duration. In view of the above, there is still a need to develop hyperbaric solution for injection of Levobupivacaine for making the spread of anaesthesia more predictable with greater spread in the direction of gravity and stable during its shelf life.

Therefore, the object of the invention is to provide a stable Hyperbaric solution for Injection of Levobupivacaine.

### SUMMARY OF THE INVENTION

In line with the above objective, the present invention provides stable hyperbaric injection solution of pharmaceutically acceptable salt of Levobupivacaine. In a preferred aspect, the invention provides a hyperbaric solution for injection composition of Levobupivacaine Hydrochloride which comprises; Levobupivacaine Hydrochloride; a baricity adjuster, a base/acid to adjust the pH together with water as a vehicle. The baricity adjuster is selected from dextrose and mannitol.

In another aspect, the invention provides process of preparation of a stable hyperbaric injection of Levobupivacaine Hydrochloride.

### DETAILED DESCRIPTION OF THE INVENTION

In accordance with the objective, the invention provides stable hyperbaric solution for injection of Levobupivacaine Hydrochloride which comprises;

Levobupivacaine Hydrochloride a baricity adjuster, a base/acid to adjust the pH together with water as a vehicle.

Levobupivacaine had shown a lower risk of cardiovascular and CNS toxicity than Bupivacaine in animal studies.

The main rational of the present invention is to provide more rapid onset and greater spread of anasthesia but a shorter duration of anaesthesia and analgesia. Thus this solution is primarily useful for abdominal surgery procedures of limited duration.

The Hyperbaric solutions have a greater specific gravity than the cerebrospinal fluid often making the spread of anaesthesia more predictable with greater spread in the direction of gravity.

According to present invention Levobupivacaine is provided in hyperbaric solution for injection for spinal anaesthesia.

In an embodiment, the instant invention provides a hyperbaric solution composition of Levobupivacaine Hydrochloride which comprises Levobupivacaine Hydrochloride; baricity adjuster, a base/acid to adjust the pH with water as a vehicle.

The baricity adjuster is selected from mannitol or dextrose.

In a preferred embodiment, the hyperbaric solution for injection composition comprises Levobupivacaine Hydrochloride; a base/acid to adjust the pH; Dextrose and water as a vehicle.

The hyperbaric solution injection composition according to the invention contains 2% w/v to 25% w/v of Dextrose.

In an embodiment, the hyperbaric solution injection may preferably contains 4% w/v to 15% w/v of Dextrose.

According an embodiment, the pH of hyperbaric solution injection is adjusted with base/acid like potassium hydroxide, sodium hydroxide/Glacial acetic acid, hydrochloric acid.

In a preferred embodiment, the pH of the hyperbaric solution injection may preferably be adjusted with base/acid like sodium hydroxide/ Hydrochloric acid.

According to the embodiment, the pH of the hyperbaric solution injection is maintained between 4.0 to 6.0.

According to the preferred embodiment, the pH of hyperbaric solution injection is maintained between 4.5 to 6.0.

In another preferred embodiment, the hyperbaric solution for injection composition comprises Levobupivacaine hydrochloride, a base/acid to adjust the pH; mannitol and water as a vehicle.

In another preferred embodiment, the invention provides a process for preparation of hyperbaric solution injection composition which comprises:
a) Dissolving Dextrose/mannitol in water for injections, followed by addition of Levobupivacaine Hydrochloride;
b) Adjusting the pH of the solution between 4.5 to 6.0; and
c) Making the required volume with cool water for injections.

The hyperbaric solution according to the invention contains 2% w/v to 25% w/v of Dextrose.

In an embodiment, the hyperbaric solution may preferably contain 4% w/v to 15% w/v of Dextrose. According to the process the pH of the hyperbaric solution is adjusted with solution of Sodium Hydroxide or Hydrochloric acid.

A hyperbaric solution for injection composition of Levobupivacaine Hydrochloride according to invention comprises 4% w/v to 15% w/v of Dextrose to make the solution hyperbaric.

A hyperbaric solution for injection composition of Levobupivacaine Hydrochloride according to the invention, wherein, the 4% w/v to 15% w/v of Dextrose is dissolved in water for injections.

In another embodiment, the hyperbaric solution for injection composition of Levobupivacaine according to the invention contains 5% w/v to 15% w/v of mannitol.

In a preferred embodiment, the hyperbaric solution may preferably contain 5% w/v to 10% w/v of mannitol.

The hyperbaric solution of Levobupivacaine Hydrochloride according to the invention, wherein, 5% w/v to 10% w/v of mannitol is dissolved in water for injection to make the solution hyperbaric.

Several different trials were conducted & tested for stability by subjecting the hyperbaric solution of Levobupivacaine Hydrochloride prepared in accordance with the invention to accelerated degradation studies (40° C.±2° C./75% RH±5% RH). Some of these trials are discussed below in brief.

The following examples, which include preferred embodiments, will serve to illustrate the practice of this invention, it being understood that the particulars shown are by way of examples and for purpose of illustrative discussion of preferred embodiments of the invention.

### EXAMPLES

### Example 1

| Ingredient | Quantity/mL |
|---|---|
| Levobupivacaine Hydrochloride | 5 mg |
| Mannitol | 5% w/v |
| Sodium Hydroxide/Hydrochloric | q.s. to pH 4.5 to 6.0 |
| acid | |
| Water For Injections | q.s. to 1 ml |

Procedure:
a) Dissolving Mannitol in a cool water for injections, followed by addition of Levobupivacaine Hydrochloride,
b) adjusting pH of the solution to 4.5 to 6.0 with solution of Sodium Hydroxide or Hydrochloric acid; and
c) Making up the required volume with cool water for injections.

The results are discussed in table 1 herein below:

| | | | Chromatography Impurity | |
|---|---|---|---|---|
| | | | Individual | Total |
| | | | impurity | Impurity |
| Stage | Assay % | Baricity | N.M.T. 0.5% | N.M.T. 1.0% |
| Initial | 103.65 | Hyperbaric | Nil | Nil |
| 1M/25° C. | 103.81 | Hyperbaric | Nil | Nil |
| 2M/25° C. | 106.83 | Hyperbaric | Nil | Nil |
| 3M/25° C. | 102.58 | Hyperbaric | Nil | Nil |
| 1M/40° C. | 102.29 | Hyperbaric | Nil | Nil |
| 2M/40° C. | 101.31 | Hyperbaric | Nil | Nil |
| 3M/40° C. | 106.64 | Hyperbaric | Nil | Nil |

### Example 2

| Ingredient | Quantity/mL |
|---|---|
| Levobupivacaine Hydrochloride | 5 mg |
| Mannitol | 9% w/v |
| Sodium Hydroxide/Hydrochloric | q.s. to pH 4.5 to 6.0 |
| acid | |
| Water For Injections | q.s. to 1 ml |

Procedure:
a) Dissolving Mannitol in a cool water for injections, followed by addition of Levobupivacaine Hydrochloride,
b) adjusting pH of the solution to 4.5 to 6.0 with solution of Sodium Hydroxide or Hydrochloric acid and
c) Making up the required volume with cool water for injections.

The results are discussed in table 2 herein below:

| | | | Chromatography Impurity | |
|---|---|---|---|---|
| | | | Individual | Total |
| | | | impurity | Impurity |
| Stage | Assay % | Baricity | N.M.T. 0.5% | N.M.T. 1.0% |
| Initial | 102.69 | Hyperbaric | Nil | Nil |
| 1M/25° C. | 104.38 | Hyperbaric | Nil | Nil |
| 2M/25° C. | 103.75 | Hyperbaric | Nil | Nil |
| 3M/25° C. | 101.56 | Hyperbaric | Nil | Nil |
| 1M/40° C. | 103.93 | Hyperbaric | Nil | Nil |
| 2M/40° C. | 102.73 | Hyperbaric | Nil | Nil |
| 3M/40° C. | 102.87 | Hyperbaric | Nil | Nil |

### Example 3

| Ingredient | Quantity/mL |
|---|---|
| Levobupivacaine Hydrochloride | 5 mg |
| Dextrose | 5.5% w/v |
| Sodium Hydroxide/Hydrochloric | q.s. to pH 4.5 to 6.0 |
| acid | |
| Water For Injections | q.s. to 1 ml |

Procedure:
a) Dissolving Dextrose in a cool water for injections, followed by addition of Levobupivacaine Hydrochloride,
b) adjusting pH of the solution to 4.5 to 6.0 with solution of Sodium Hydroxide or Hydrochloric acid;and
c) Making up the required volume with cool water for injections.

The results are discussed in table 3 herein below:

| | | | Chromatography Impurity | |
|---|---|---|---|---|
| | | | Individual | Total |
| | | | impurity | Impurity |
| Stage | Assay % | Baricity | N.M.T. 0.5% | N.M.T. 1.0% |
| Initial | 102.35 | Hyperbaric | Nil | Nil |
| 1M/25° C. | 103.89 | Hyperbaric | Nil | Nil |
| 2M/25° C. | 102.67 | Hyperbaric | Nil | Nil |
| 3M/25° C. | 102.80 | Hyperbaric | Nil | Nil |
| 1M/40° C. | 103.92 | Hyperbaric | Nil | Nil |
| 2M/40° C. | 103.25 | Hyperbaric | Nil | Nil |
| 3M/40° C. | 101.23 | Hyperbaric | Nil | Nil |

### Example 4

| Ingredient | Quantity/mL |
|---|---|
| Levobupivacaine Hydrochloride | 5 mg |
| Dextrose | 8% w/v |
| Sodium Hydroxide/Hydrochloric | q.s. to pH 4.5 to 6.0 |
| acid | |
| Water For Injections | q.s. to 1 ml |

Procedure:
a) Dissolving Dextrose in a cool water for injections, followed by addition of Levobupivacaine Hydrochloride,
b) adjusting pH of the solution to 4.5 to 6.0 with solution of Sodium Hydroxide or Hydrochloric acid; and
c) Making up the required volume with cool water for injections.

The results are discussed in table 4 herein below:

| | | | Chromatography Impurity | |
|---|---|---|---|---|
| | | | Individual | Total |
| | | | impurity | Impurity |
| Stage | Assay % | Baricity | N.M.T. 0.5% | N.M.T. 1.0% |
| Initial | 106.22 | Hyperbaric | Nil | Nil |
| 1M/25° C. | 106.94 | Hyperbaric | Nil | Nil |
| 2M/25° C. | 102.97 | Hyperbaric | Nil | Nil |
| 3M/25° C. | 101.90 | Hyperbaric | Nil | Nil |
| 1M/40° C. | 106.61 | Hyperbaric | Nil | Nil |
| 2M/40° C. | 103.97 | Hyperbaric | Nil | Nil |
| 3M/40° C. | 100.49 | Hyperbaric | Nil | Nil |

### Example 5

| Ingredient | Quantity/mL |
|---|---|
| Levobupivacaine Hydrochloride | 5 mg |
| Dextrose | 10% w/v |
| Sodium Hydroxide/Hydrochloric | q.s. to pH 4.5 to 6.0 |
| acid | |
| Water For Injections | q.s. to 1 ml |

Procedure:
a) Dissolving Dextrose in a cool water for injections, followed by addition of Levobupivacaine Hydrochloride,
b) adjusting pH of the solution to 4.5 to 6.0 with solution of Sodium Hydroxide or Hydrochloric acid and
c) Making up the required volume with cool water for injections.

The results are discussed in table 5 herein below:

| | | | Chromatography Impurity | |
|---|---|---|---|---|
| | | | Individual | Total |
| | | | impurity | Impurity |
| Stage | Assay % | Baricity | N.M.T. 0.5% | N.M.T. 1.0% |
| Initial | 103.28 | Hyperbaric | Nil | Nil |
| 1M/25° C. | 106.89 | Hyperbaric | Nil | Nil |
| 2M/25° C. | 102.45 | Hyperbaric | Nil | Nil |
| 3M/25° C. | 102.54 | Hyperbaric | Nil | Nil |
| 1M/40° C. | 104.39 | Hyperbaric | Nil | Nil |
| 2M/40° C. | 103.74 | Hyperbaric | Nil | Nil |
| 3M/40° C. | 102.64 | Hyperbaric | Nil | Nil |

### Example 6

| Ingredient | Quantity/mL |
|---|---|
| Levobupivacaine Hydrochloride | 5 mg |
| Dextrose | 12.5% w/v |
| Sodium Hydroxide/Hydrochloric | q.s. to pH 4.5 to 6.0 |
| acid | |
| Water For Injections | q.s. to 1 ml |

Procedure:
a) Dissolving Dextrose in a cool water for injections, followed by addition of Levobupivacaine Hydrochloride,
b) adjusting pH of the solution to 4.5 to 6.0 with solution of Sodium Hydroxide or Hydrochloric acid and
c) Making up the required volume with cool water for injections.

The results are discussed in table 6 herein below:

| | | | Chromatography Impurity | |
|---|---|---|---|---|
| | | | Individual | Total |
| | | | impurity | Impurity |
| Stage | Assay % | Baricity | N.M.T. 0.5% | N.M.T. 1.0% |
| Initial | 102.21 | Hyperbaric | Nil | Nil |
| 1M/25° C. | 103.54 | Hyperbaric | Nil | Nil |
| 2M/25° C. | 104.25 | Hyperbaric | Nil | Nil |
| 3M/25° C. | 106.23 | Hyperbaric | Nil | Nil |
| 1M/40° C. | 105.21 | Hyperbaric | Nil | Nil |
| 2M/40° C. | 102.45 | Hyperbaric | Nil | Nil |
| 3M/40° C. | 102.87 | Hyperbaric | Nil | Nil |

1-19. (canceled)

20. A stable hyperbaric injection solution of Levobupivacaine, comprising;
a) a pharmaceutically acceptable salt of Levobupivacaine;
b) a baricity adjuster to make the solution hyperbaric;
c) a pH adjuster selected from the group consisting of a base, an acid, and a combination thereof; and
d) a vehicle.

21. The stable hyperbaric injection solution of claim 20, wherein the baricity adjuster is Mannitol, Dextrose, or a mixture thereof.

22. The stable hyperbaric injection solution of claim 21; wherein the baricity adjuster is Dextrose.

23. The stable hyperbaric injection solution of claim 22, wherein the Dextrose is present in an amount of 2% w/v to 25% w/v.

24. The stable hyperbaric injection solution of claim 23, wherein the Dextrose is present in an amount of 4% w/v to 15% w/v.

25. The stable hyperbaric injection solution of claim 21, wherein the baricity adjuster is Mannitol.

26. The stable hyperbaric injection solution of claim 25, wherein the Mannitol is present in an amount of 5% w/v to 10% w/v.

27. The stable hyperbaric injection solution of claim 20, wherein the pH adjuster is selected from the group consisting of potassium hydroxide, sodium hydroxide, Glacial acetic acid, hydrochloric acid, or a mixture thereof.

28. The stable hyperbaric injection solution of claim 22, wherein the pH of the solution is between 4.5 and 6.0.

29. The stable hyperbaric injection solution of claim 20, wherein the pharmaceutically acceptable salt of Levobupivacaine is a hydrochloride salt of Levobupivacaine.

30. A process for preparation of a stable hyperbaric injection solution, comprising:
a) forming a solution by dissolving a baricity adjuster in water, followed by addition of a pharmaceutically acceptable salt of Levobupivacaine;
b) adjusting the pH of the solution to between 4.5 and 6.0; and
c) adding a required volume of water to the solution to obtain the hyperbaric injection solution.

31. The process of claim 30, wherein the process involves addition of a hydrochloride salt of Levobupivacaine.

32. The process of claim 30, wherein the baricity adjuster is Dextrose; and the Dextrose is used in an amount of 2% w/v to 25% w/v.

33. The process of claim 32, wherein the Dextrose is used in an amount of 4% w/v to 15% w/v.

34. The process of claim 30, wherein the baricity adjuster is Mannitol; and the Mannitol is used in an amount of 5% w/v to 10% w/v.

35. The process of claim 30, wherein the pH is adjusted with sodium hydroxide, hydrochloric acid, or a mixture thereof.

36. A stable hyperbaric injection solution of Levobupivacaine, comprising;
a) a pharmaceutically acceptable salt of Levobupivacaine;
b) 2% w/v to 25% w/v of Dextrose to make the solution hyperbaric;
c) a pH adjuster selected from the group consisting of a base, an acid, and a combination thereof; and
d) an aqueous vehicle.
